(19) United States
(12) Patent Application Publication
(10) Pub. No.: US 2013/0109933 A1
(43) Pub. Date: May 2, 2013

(54) IMPLANT

(71) Applicant: BIOTRONIK AG, Buelach (CH)

(72) Inventors: Eric Wittchow, Nuernberg (DE); Olaf Skerl, Bad Doberan (DE); Gerald Czygan, Buckenhof (DE)

(73) Assignee: BIOTRONIK AG, Buelach (CH)

(21) Appl. No.: 13/660,971

(22) Filed: Oct. 25, 2012

Related U.S. Application Data
(60) Provisional application No. 61/551,941, filed on Oct. 27, 2011.

(51) Int. Cl. A61B 5/103; A61B 5/07

(52) U.S. Cl. 600/302; 600/587

(57) ABSTRACT

An implant, having a preferably hollow-cylindrical base body and comprising a device that is used to measure the degree of endothelialization and disposed on and/or in the base body, wherein the device comprises an acoustic resonator and/or an electric resonator. The invention further relates to a system, comprising a catheter and such an implant.

### CROSS REFERENCE TO RELATED APPLICATIONS

This application claims benefit of priority to U.S. provisional patent application Ser. No. 61/551,941, filed Oct. 27, 2011; the contents of which are herein incorporated by reference in their entirety.

### TECHNICAL FIELD

The present invention relates to an implant, and more particularly to an intraluminal endoprosthesis, having a base body that is optionally hollow and cylindrical, and to a system comprising a catheter and such an implant.

### BACKGROUND

Medical endoprosthesis or implants for a wide variety of applications are known from the state of the art in great diversity. Implants as defined by the present invention shall be endovascular prostheses or other endoprostheses, such as stents (vascular stent (including the heart and heart valve stents), bile duct stent, mitral stent), endoprostheses for closing patent foramen ovale (PFO), pulmonary valve stents, endoprosthesis for closing an atrial septal defect (ASD), and prostheses in the area of hard and soft tissues.

These days, stents that are used for the treatment of stenoses (vascular constrictions) are employed especially frequently as implants. They generally have a tubular or hollow-cylindrical base body, which is open at both longitudinal ends and typically perforated. In many cases, the base body of the stent is composed of individual mesh sections, which are formed of struts having various shapes, for example zigzag- or meander-shaped struts. Such an endoprosthesis is generally inserted into the vessel requiring treatment by means of a catheter and, is intended to support the vessel over an extended time period (months to years). Constricted areas in the vessels can be dilated through the use of stents, resulting in increased lumen. While through the use of stents or other implants, an optimal vessel cross-section can be achieved, which is primarily necessary for a successful treatment, the lasting presence of a stent, which constitutes a foreign object per se, triggers a cascade of microbiological processes, which favor inflammation of the vessel to be treated or necrotic vascular changes, for example, and/or may result in gradual blockage of the stent due to the formation of plaque or coagulation of body fluid resulting from a change in flow or resulting from inflammation or infection processes.

So as to avoid restenoses or inflammations and necroses, stents or other implants can be provided with a coating of drugs or other pharmaceutically active substances, which have an anticoagulant, antiproliferative or anti-inflammatory effect, for example. Such stents are also referred to as drug-eluting stents (hereinafter in short: DES).

The introduction of DES, which elute paclitaxel or sirolimus, for example, has allowed a considerable decrease in the restenosis rate as compared to stents having no coating. This is leads to cost savings because the necessity for revascularization drops significantly.

The enthusiasm for the new treatment options, however, has been curbed by an unchanged or even slightly higher number of late stent thromboses (hereinafter in short: LST). Meta-analyses show that the treatment with DES is accompanied by a rising percentage of LST. In patients with particularly unfavorable conditions, the LST rate may rise to as much as 8.2%. Such thromboses, however, must be prevented to as great an extent as possible, because they frequently have a fatal outcome (20 to 40%), can result in myocardial infarction (50 to 70%) and often necessitate revascularization.

In addition to infrequently occurring hypersensitivity, theories presently under discussion cite a delayed healing process, which manifests itself in persistent fibrin deposition and incomplete endothelialization, as the primary cause of DES-related late thromboses. In the case of incomplete endothelialization, the non-endothelialized stent struts can be the cause for thrombosis. This hypothesis is primarily based on autopsy studies, in which delayed healing was observed in all LST cases. Current guidelines therefore recommend a dual anticoagulation therapy lasting several months by means of aspirin and clopidogrel with the implantation of a DES, whereby the entire treatment becomes considerably more expensive. Moreover, such a therapy also leads to interactions with other therapies, notably with surgical procedures.

The optimal duration of such an anticoagulation therapy is not clear at present. Follow-up examinations of the healing process could provide conclusions in this respect. It is therefore desirable to monitor the progression of endothelialization during DES therapy.

A stent, which comprises at least one cantilever in the stent mesh, is known from the document “Sensor to detect endothelialization on an active coronary stent”, K. M. Musick et al., BioMedical Engineering OnLine 2010, 9:67. This cantilever contains a piezo element comprising a film of zinc oxide. Populating the cantilever with body cells (endothelialization) alters the cantilever's resonance frequency and thereby allows the degree of endothelialization in vivo to be measured. The known solution, however, is disadvantageous with respect to the dimensions thereof, because such a cantilever should neither protrude into the vessel nor significantly alter the design of the stent body, because this body could otherwise trigger proliferation or cause thrombi as a result of changes in laminar flow. Moreover, the functionality and electronics do not meet the requirements for a use in the field of implants. In terms of the functionality, it should be noted that the resonance frequency of a cantilever oscillating in a fluid is influenced not only by the increase in the mass thereof, but also significantly by the properties of the surrounding fluid (for example the viscosity thereof, flow rate). Both effects cannot be separated from each other, whereby reliable determination of the increase in mass cannot be assured. In terms of the electronics, this prior art explains that the sensor is designed as an active sensor, which contains electronic elements for generating readings and for data transfer purposes, in addition to the transducer per se. The required auxiliary power should likewise be supplied wirelessly from the outside. The resulting size of the electronics unit of the solution according to the prior art, which is required in addition to the transducer per se, is not acceptable.

### SUMMARY

The object is thus to create an implant which allows endothelialization to be monitored, without requiring significant changes to the outside dimensions and the structure of the implant.

The above object is achieved by an implant comprising a device (hereinafter also referred to as microsensor) which is used to measure the degree of endothelialization and is disposed on and/or in the base body, the device comprising an acoustic resonator and/or an to electric resonator.

The implant according to the invention, comprising the device capable of and thus for measuring the degree of endothelialization, which has an acoustic resonator and/or an electric resonator, allows the degree of population of the implant with endothelial cells to be determined in a simple manner. In the case of delayed endothelialization, the anticoagulation therapy can be extended individually until coverage of the implant with tissue is complete during repeat measurement. The risk of LST can thus be significantly reduced. Conversely, if the stent is covered more quickly by endothelial tissue, the anticoagulation therapy can be discontinued sooner than pursuant to the present procedure having no follow-up examination, whereby treatment costs can be saved and unnecessary side effects with other therapies may occur only over a short time period. The advantage of the implant according to the invention is therefore in particular that of being able to discontinue medication after complete endothelialization. In other words, the present invention can prevent the anticoagulation therapy from being discontinued prior to complete endothelialization.

It is possible today to implement an acoustic resonator, or an electric resonator, in such a small size that the microsensor does not significantly alter the dimensions of the implant. The microsensor is preferably disposed in and/or on the luminal side of the implant.

The microsensor is preferably designed as a purely passive sensor, which contains no additional electronic devices and requires no auxiliary power to function. This also makes extreme miniaturization possible.

Within the scope of the present invention, the degree of endothelialization shall be understood to mean the rate of tissue coverage on the sensor that forms on the surface of the implant after insertion thereof. If the surface of the implant is entirely covered by tissue, the tissue coverage, and hence the degree of endothelialization, is 100%. In the case in which the device comprises an acoustic resonator, the degree of endothelialization is determined by measuring the mass adhering to the implant, while in the case in which the device comprises an electric resonator, the degree of endothelialization is determined by measuring the covered surface area.

Immediately after implantation, the surface of the implant does not contain any endothelial tissue. In rabbit models, uncoated stents are fully endothelialized after approximately 14 days, while stents containing antiproliferative drugs are endothelialized after approximately 4 weeks. In humans, growth takes place considerably more slowly, with estimates ranging around a factor of 4 to 6. One human study analyzing the population of stents based on autopsy dissections shows that the uncoated implants are endothelialized approximately one third after 3 weeks. (Anderson P. G., Bajaj R. K., Baxley W. A., Roubin G. S., “Vascular patholgy of balloon-expandable flexible coil stents in human”, JACC 1992, 19, pages 372 to 381).

The current recommendations of the consensus group “European Society of Cardiology Working Group on Thrombosis” in terms of the duration of a dual anticoagulation therapy are 1 month for an uncoated stent, at least 3 months for a stent eluting sirolimus, everolimus or tacrolimus, and 6 months for a paclitaxel-eluting stent. A longer duration may be considered for select patients at low risk of bleeding (source: Lip G. Y., Huber K., Andreotti F., Arnesen H., Airaksinen K. J., Cuisset T., Kirchhof P., Marin F., “Management of antithrombotic therapy in atrial fibrillation patients presenting with acute coronary syndrome and/or undergoing percutaneous coronary intervention/stenting”, Thromb Haemost 2010, 103, pages 13-28).

Endothelium denotes the layer of cells that lines the innermost wall layer of lymphatic and blood vessels (tunica intima) and faces the vessel lumen.

The acoustic and/or electric resonator is notably disposed in and/or on the implant such that a change in the mass and/or in the surface area of the coverage of the surface of the device by endothelial cells affects a corresponding change in the electric output signal of the device. The acoustic resonator and/or the electric resonator are preferably disposed on the inside (luminal side) of the implant, wherein still more preferably a section of the to respective resonator is exposed on the surface. Exposing the sections of the respective device causes the endothelial cells to grow directly on the surface of the device, whereby very exact results in terms of the endothelialization can be achieved.

Still more preferably, the acoustic resonator comprises at least one SAW (surface acoustic wave) element. Such an element operates based on surface acoustic waves, these being structure-borne sound waves propagating in a planar manner on the surface of the element, which is to say in two dimensions. Such a SAW element utilizes the dependence of the surface acoustic wave velocity on the adherence of mass in the region of the surface of the SAW element, which is to say the adhering mass of body cells, such as endothelial cells, on the surface of the SAW element. Still more preferably, a SAW element that operates based on shear horizontal waves (SH-SAW) is used.

This exemplary embodiment takes advantage of the property of the shear horizontal waves having an oscillation plane parallel to the surface that they couple into the fluid (for example the liquid in the vessel) only minimally and consequently the wave propagation is influenced only little by the liquid. Even a “loose” cell located on the surface would interfere little with the wave propagation. Only cells that are grown to the surface, which form the desired endothelialization, affect a considerable change in the wave propagation, and thus a decrease in the wave propagation velocity as well as, consequently, a decrease in the resonant frequency or an increase in the propagating time. Compared to the solution according to the aforementioned prior art comprising a cantilever, endothelialization can thus be determined much more precisely by means of the implant according to the invention.

In an alternative exemplary embodiment, the electric resonator comprises at least one capacitor and at least one coil, wherein the total capacitor (optionally comprising capacitors connected in parallel) and the coil are connected in series and form an oscillating circuit, the resonant frequency of which depends on the total capacitance of the one capacitor, or of the plurality of capacitors connected in parallel. The changing population of the implant with body cells during healing following the implantation, which to is to say the accordingly changing volume of the endothelial cells present in the stray field of the electrodes of the at least one capacitor, reduces the stray capacitance between the electrodes and thus raises the resonant frequency of the electric resonator. The coil and the at least one capacitor can be attached either to the luminal side of the implant or designed, either individually or collectively, as part of the implant, for example the stent struts can be designed as electrodes/coils. The electrodes of the capacitor can preferably be designed as strip electrodes on a suitable film that covers a part of the luminal surface area of the implant.

This exemplary embodiment also advantageously utilizes of the property of the capacitor that the stray field formed by the electrodes of the capacitor responds with great sensitivity to the distance of the body cells from the surface of the implant (proportional to 1/(distancê2)). The greater the distance of a body cell to the electrode plane (surface of the implant) or the device, the lower is the influence thereof on the stray field, and hence on the resonant frequency of the resonator. Consequently, notably the body cells that are attached directly to the surface of the implant, which is to say the endothelialization, are also measured in this exemplary embodiment, whereby a crucial advantage over the prior art is achieved.

The capacitance of the capacitor preferably ranges between 5 pF and 20 pF, and the inductance of the coil preferably ranges between 200 nH and 500 nH. The resulting resonant frequency of the oscillating circuit thus ranges between 50 MHz and 200 MHz in the preferred exemplary embodiment.

It is further advantageous for the surface of the device, and more particularly the SAW element of the acoustic resonator and/or the capacitor of the electric resonator, to comprise at least one coating. This coating may be a passivation layer and/or a coating that exhibits the same, or at least similar, population properties as the base body of the implant. A coating that is insulating and thus prevents the electrodes of the capacitor from being short-circuited is advantageous notably for the capacitor of an electric resonator.

In particular a polymer material that is stable in the blood vessel over a long period of time and incites the least tissue reaction possible, and that additionally does not tend to form thrombi, is suitable for such a coating. A thin film made of Parylene (for example Parylene-C) is particularly suited, which is applied by means of a plasma method. Other inert polymers such as polyurethanes, silicones, Teflon or acrylates are also suitable.

The layer thickness of the coating over the electrode preferably ranges between 1μ and 10 μm, with layers that predominantly comprise Parylene still more preferably having layer thicknesses between 1 μm and 3 μm. Coatings comprising other materials still more preferably range between 5 μm and 10 μm.

So as to enable wireless scanning of the signals generated by the device for measuring endothelialization by means of an appropriate device for evaluating the signals, the device comprises at least one antenna, which is connected to the acoustic resonator and/or the electric resonator. To this end, for example, a stent strut can be designed as an antenna or comprises an antenna and/or can be applied to or introduced in the base body of the implant. The at least one antenna is still more preferably attached to the vessel side of the implant, which is to say to the outside or the abluminal side, so that it is not shielded by the implant (as in a Faraday cage). As an alternative, it is also possible for it to protrude over the implant at both ends in the direction of the longitudinal axis.

Still more preferably, the base body of the implant and the antenna comprise the same material or consist of the same material. Cobalt chromium steels (L605, MP35N) are particularly suited, as are surgical stainless steel (316L) or nickel titanium steels (nitinol).

Optionally, poor biocompatibility of an antenna material may be improved by using a polymeric coating (for example by means of Parylene-C).

The microsensor is activated by a scanning unit by means of this at least one antenna, the scanning unit being equipped with at least one corresponding transmitting antenna and at least one corresponding receiving antenna. The scanning unit and/or the processor integrated therein, or connected thereto, calculate the degree of endothelialization based on the signals received from the microsensor and display this degree, or transmit it to a database connected to the processor. Using the endothelialization data from previous time periods that is already stored in the database, the progression of endothelialization can thus be determined and optionally displayed.

In the exemplary embodiment in which an acoustic resonator is employed, an RF wave, for example, can be conducted to an acoustic resonator comprising an interdigital electrode is (IDT), which is disposed on a piezoelectric material. The IDT generates an acoustic wave packet from this. The propagation of this wave packet, which is preferably a shear horizontal wave, in the implant is monitored and the mass of the endothelial cell layer on the surface of the implant, and thus the degree of endothelialization, are determined, for example, based on the frequency shift of the resonator and/or the attenuation of the signal and/or the decrease in the quality of the resonator. For this purpose, a delay line sensor or a one port sensor may be used in the device.

When using an electric resonator, for example, the resonant frequency of the oscillating circuit of the device, which depends on the total capacitance that varies as a result of the endothelialization, is determined by means of a scanning unit designed as a spectrum analyzer.

In the exemplary embodiment of the invention, the implant comprises a pharmaceutically active substance on at least a portion of the surface of the base body.

Within the scope of the present invention, a pharmaceutically active substance (or therapeutically active or effective substance) shall be a plant, animal or synthetic active ingredient (drug) or a hormone, which in a suitable dose is used as a therapeutic agent for influencing states or functions of the body, for substituting active ingredients produced naturally by the human or animal body, such as insulin, and for eliminating, or rendering harmless, pathogens, tumors, cancer cells or substances foreign to the body. The release of the substance in the surroundings of the implant has a positive effect on the healing process or counteracts pathological changes of the tissue as a result of the surgical procedure, or in oncology is used to render diseased cells harmless.

Such pharmaceutically active substances, for example, have an anti-inflammatory and/or antiproliferative and/or spasmolytic effect, whereby, for example, restenoses, inflammations or (vascular) spasms can be avoided. In particularly preferred exemplary embodiments, such substances may comprise one or more substances of the active substances groups consisting of the calcium channel blockers, lipid regulators (such as fibrates), immunosuppressants, calcineurin inhibitors (such as tactrolimus), antiphlogistics is (such as cortisone or dichlofenac), anti-inflammatory agents (such as imidazoles), anti-allergic drugs, oligonucleotides (such as dODN), estrogens (such as genistein), endothelial forming agents (such as fibrin), steroids, analgesics, antirheumatism agents, proteins, hormones, insulins, cytostatic drugs, peptides, vasodilators (such as sartanes), and the antiproliferatively (proliferation-inhibiting) acting substances of the taxols or taxanes, preferably paclitaxel or sirolimus, or may be taken from the following list: cisplatin, tirapazamine, enzyme L-asparaginase, methotrexate, 5-fluorouracil, azathioprine, mitoxantrone, cyclophosphamide, methotrexate, natalizumab, adriamycin PFS, adriamycin RDF, alitretinoin, altretamine, aromasin, azathioprine, bicalutamide, busulfan, busulfex, capecitabine, casodex, cyclophosphamide, cytoxan, doxorubicin, exemestane, femara, finasteride, gemtuzumab, ozogamicin, hexalen, imuran, letrozole, mifeprex, mifepristone, myleran, mylotarg, neosar, nolvadex, panretin, propecia, proscar rubex, tamoxifen, temodar, temozolomide, trelstar depot, triptorelin, genasense (Genta), INGN201 (Introgen Therapeutics), SCH58500 (Schering-Plough), ONYX-015 (Onyx Pharmaceuticals), E1A—lipid complex (Targeted Genetics), TRAIL (Genentech/Immunex), GX01 (Gemin X Biotechnologies), cyclosporin A, DPPE, PSC 833, buthionine sulphoximine, dexverapamil, quinine, verapamil, XR9576, dexniguldipin, GF120918, lobradimil, LY335979, MS209, R-101933, gemtuzumab ozogamicin, SGN-15, MCC-465, SB-408075, A5B7 antibody against CEA with carboxy peptidase A+mustard prodrug, amifostine, dexrazoxane, BB-10010, transfer of MDR genes, BNP7787, tirapazamine, aplidine, arsenic trioxide, BMS-247550, CHS828, CT 2584, dolastatin-10, ET-743, exisulind, irofulven, KW-2189, lovastatin, E7070, LU103793, LY355703, pyrazoloacridine, TLK286, apomine, CP-461, EP0906, FB642, FK317, FK866, kahalalide F, LAF389, PNU-166196, RO 31-7453, cetuximab (Erbitux), trastuzumab (Herceptin), ABX-EGF, AP12009, EMD55900, EMD72000, ICR62, 2A11, CCI-779, ISIS-3521, oblimersen (Genasense), OSI-774 (Tarceva), PS-341, R115777 (Zarnestra), STI571 (Gleevec), ZD1839 (Iressa), bryostatin-1, flavopiridol, GD0039, GEM231, ilmofosine, ISIS-2503, ISIS-5132, L-778 123, PKC 412, SCH66336, SU-101, UCN-01, Bay 43-9006, BMS-214662, CI-1040, GW572016, LErafAON, LY-317615, perifosine, phenoxodiol, PKI 166, swainsonine, 17-AAG, decitabine, CI-994, depsipeptide, MG98, phenylbutyrate, phenylacetate, suberoylanilidehydroxamic acid, Adp53, antineoplastons, A10/AS2-1, OL(1)p53, p53, RPR/INGN-201, SCH 58500, HSV-TK VPC, tgDCC-E1A, INX3280, TK gene pioglitazone, troglitazone, BAY12-9566, BMS-275291, clodronate, marimastat, prinomastat, MMI270, COL-3, CP-471,358, trans retinoic acid, bexarotene, pivaloyloxy-methylbutyrate, 9-cis-retinoic acid, 13-cis RA, fenretinide, ILX23-7553, TAC-101, tazarotene, bevacizumab, RhuMab-VEGF, HuMV833, angiozyme, IMC-1C11, PI-88, SU5416, CP547,632, PNU-145156E, PTK/ZK 787, SU6668, ZD6474, carboxyamidotriazole, GBC-590, squalamine, vitxain, ABT-510, CM101, ZD6126, neovastat, suramin, thalidomide, IM862, TNP-470, angiostatin, CC-5013, combretastatin A4, endostatin, interleukin-12, alemtuzumab, edrecolomab, epratuzumab HuM195, oregovomab, rituximab, Ch14.18, MDX-11, WX-G250, 3F8, H22xKI-4, ING-1, J591, KM871, immunoconjugates antibody with toxin, BL22, Anti-Tac-PE38 (LMB-2), BB-10901, SS1-PE38, denileukin diftitox (ONTAK), IL13-PE38QQR, TP-38, Allovectin-7, 105AD7, BEC2, TriGem, 1A7, 3H1, vaccines, MDX-H210, G17DT, MDX-447, EMD 273063, IL-2/histamine, LAK, TIL, CTL, Bay 50-4798, MDX-010, OK-432, PSK, ubenimex, GM-CSF, ONYX-015, NV1020, PV701, reolysin, celecoxib, lyprinol, LY293111, astrasentan, melatonin, taurolidine, cyclosporin A, verapamil, tirapazamine trastuzumab, clodronate, trans retinoic acid, edrecolomab, rituximab, OK-432, ubenimex, melatonin, PSC 833, R115777, ZD1839, SCH 66336, decitabine, HSV-TK, VPC, BAY12-9566, marimastat, prinomastat, suramin, 105AD7, IL-2/histamine, astrasentan.

The base body of an implant according to the invention can preferably comprise at least one element and/or a compound of the following group consisting of metals, metal alloys, preferably stainless steel, CoCr steels, magnesium alloys, iron alloys, zinc alloys, manganese alloys, nitinol, polymers from the category of biodegradable polymers, preferably polylactic acids, polyglycolic acids, polycaprolactone, mixtures or copolymers thereof, and polymers from the category of biocompatible polymers, preferably UHMWPE and PEEK. An implant is referred to as an absorbable metal stent (AMS) when it is designed as a stent and comprises a biodegradable magnesium alloy, iron alloy, zinc alloy to or manganese alloy.

The above object is further achieved by a system, comprising a catheter having a balloon and an implant, wherein the implant is described above and the implant is disposed on the, preferably folded, balloon of the catheter. Such a system is suitable for easily introducing the implant, with the aforementioned advantages, for treatment into an organism. After the catheter, together with the implant, has been placed in the desired location, the balloon is deployed during implantation and inflated, whereby the implant is dilated. When the necessary dilation, which preferably is also used to expand the surrounding vessel section, the balloon is deflated again and the catheter can be removed, while the implant remains at the treated site in the organism.

The implant according to the invention and the system according to the invention will be described hereafter in examples based on figures. All characteristics described and/or illustrated form the subject matter of the invention, regardless of their summarization in the claims or dependent claims.

### DESCRIPTION OF THE DRAWINGS

In the drawings:

FIG. 1 shows a perspective side view of a section of a first exemplary embodiment of an implant according to the invention,

FIG. 2 shows a top view of a device that is used in a second exemplary embodiment of an implant according to the invention for measuring endothelialization,

FIG. 3 shows a top view of a part of a device that is used in a third exemplary embodiment of an implant according to the invention for measuring endothelialization,

FIG. 4 shows a schematic diagram of a system comprising an implant according to the invention, a scanning unit and a database, and

FIG. 5 is a longitudinal sectional view of the device shown in FIG. 2.

### DETAILED DESCRIPTION

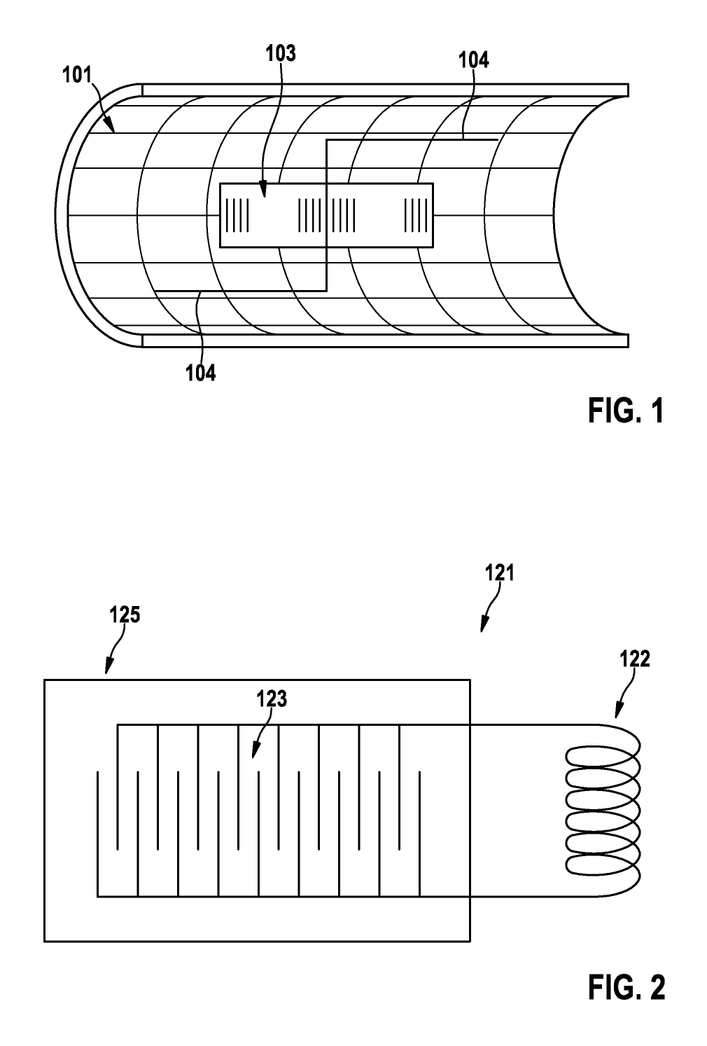

FIG. 1 shows a section of a base body 101 of an implant designed as a stent, on the luminal surface of which a SAW element 103 is disposed as part of a device for measuring the endothelialization (hereinafter in short: microsensor).

The SAW element 103 is connected to an antenna 104, which likewise forms part of the microsensor and enables wireless scanning of the signals generated by the SAW element 103. The antenna 104 is either attached to the base body 100 or designed as part of the base body 100.

The SAW element 103 forms an acoustic resonator. Preferably, what is known as a shear wave SAW element is used, which operates based on shear horizontal waves. Such a SAW element comprises, for example, an IDT, which is disposed on a piezo-electric material. This IDT generates a shear wave, the propagation of which in the implant is monitored, for example, in a design as a delay line sensor or as a one port sensor and detected by the scanning unit. The degree of endothelialization can be determined based on the shift of the resonant frequency, the decrease in quality and/or the rise in attenuation of the resonator.

A passivation layer and/or a coating, which is not shown and has identical, or at least to similar, population properties as the surface of the stent base body 100, may be provided on the surface of the SAW element 103.

The resonant frequency of the SAW element 103 ranges, for example, between 30 MHz and 5 GHz, and more preferably a resonant frequency of 400 MHz is used.

The cells adhering to the surface of the SAW element 103, which is to say the endothelial cells attaching after implantation, cause an increase in the mass adhering to the SAW element 103, which alters the acoustic properties of the SAW element 103. In a resonator, this leads, for example, to a decrease in resonant frequency and the quality and in an increase in attenuation. These parameters can be evaluated so as to determine the degree of endothelialization of the implant.

Accordingly, a scanning unit that is disposed outside of the human or animal body treated with the implant according to the invention can determine the population of the implant with body cells, this being the endothelialization, both qualitatively and quantitatively.

The scanning unit determines, for example, the resonant frequency of the SAW sensor using known methods and, based thereon, the frequency shift Δf caused by the mass loading, in relation to the unloaded state. Based on this frequency shift, the mass loading Δm of the SAW sensor, and based thereon the degree of endothelialization, are determined using

$$Δ m = - k \frac{Δ f}{{f}_{0}} , \qquad ( 1 )$$

where f₀ denotes the fundamental frequency of the SAW resonator without mass loading and k denotes a calibration constant of the sensor array.

By way of the antenna 104, the SAW element 103 can wirelessly scan the signals. This is shown in FIG. 4. The left region of FIG. 4 shows a stent 100, which is implanted in a human body 105 and on the inside of which a SAW sensor is arranged, which is scanned by means of a scanning unit 110 disposed outside of the treated body. The scanning unit 110 also comprises an antenna 112 for this purpose, which is designed in particular as a transceiver antenna. The scanning unit 110 can scan the SAW element 103 at regular intervals and receive the signals, in a manner that is controlled by the patient or the physician, for example. The scanning can also take place automatically without the involvement of the patient as soon as this patient is located in the vicinity of the scanning unit 110. In this case, the scanning unit 110 autonomously conducts and controls the scan, is which can be done once a day, for example.

In particular when the scan is carried out by means of a patient's scanning unit 110, in one exemplary embodiment of the present invention the data determined by the microsensor, or the data calculated by a processor of the scanning unit 110, such as the thickness of the endothelial tissue layer on the stent, is transmitted, preferably wirelessly, to a central database, which is part of a processor 115 or is connected thereto. The data is stored in the database, processed and made available to the treating physician. It can be displayed to the physician, for example, in the form of a tabular or graphical progression image of the thickness of the endothelial tissue layer attached to the implant, so that the population process over a defined period is available, such as one year, for example. The processor 115 can further comprise an analysis unit, or be connected to such a unit, which can deliver an automatic warning or notification to the physician when a state has developed that requires intervention.

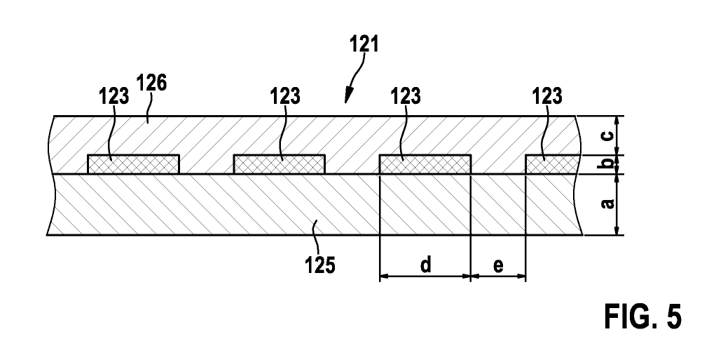

As an alternative or in addition to the SAW element 103, the microsensor may be provided with an electric resonator 121, which is shown in FIGS. 2 and 5 and comprises a coil 122 and several, mutually opposing electrodes 123, which form respective capacitors connected in parallel. The electrodes 123 can be disposed, for example, as strip electrodes on a film 125 such that the electric stray field of the electrodes penetrates the immediate vicinity of the microsensor. To this end, the electrodes are disposed on the inner (luminal) surface of the stent base body 101, so that the electrodes are exposed, except for an insulating coating. The electric conductivity of the material of the electrodes is preferably high. The electrodes can therefore comprise a metal (Ag, Au, Cu, Al, . . . ) or a conductive polymer. Such a strip electrode can, for example, have a thickness b ranging between 10 μm and 20 μm, a width d ranging between approximately 40 μm and 60 μm, a distance e of the strips to of approximately 50μ, and a length that is dependent on the stent diameter. The film 125, for example, has a thickness a ranging between 100 μm and 200 μm. The aforementioned dimensions are shown in FIG. 5.

The electrodes 123 can further be provided with a preferably insulating passivation layer and/or a coating 126, which is shown in FIG. 5 and which has the same, or at least similar, population properties as the surface of the implant body. The thickness c of the coating 126 over the electrodes 123 ranges between 1 μm and 10 μm.

The electric resonator 121, including the carrier film 125, is applied to the stent base body. In the exemplary embodiment, the stent base body is thus lined on the inside thereof (luminal side) by the carrier film 125.

The microsensor comprising the electric resonator 121 is preferably disposed on the luminal side of a stent base body. After such an implant is implanted in the body cavity, it becomes populated with body cells. This alters the stray capacitance between the electrodes 123, whereby the resonant frequency of the oscillating circuit of the electric resonator 121 changes. Analogously to the first exemplary embodiment of an implant, the resonant frequency of this oscillating circuit and the change thereof after implantation is read by means of an external scanning unit 110. Based on this, the current endothelialization of the implant, the progression thereof in the past, and the progression of the healing process can be derived. It can thus be established whether the endothelialization is progressing very slowly and whether an increased risk of late thrombosis (LST) exists.

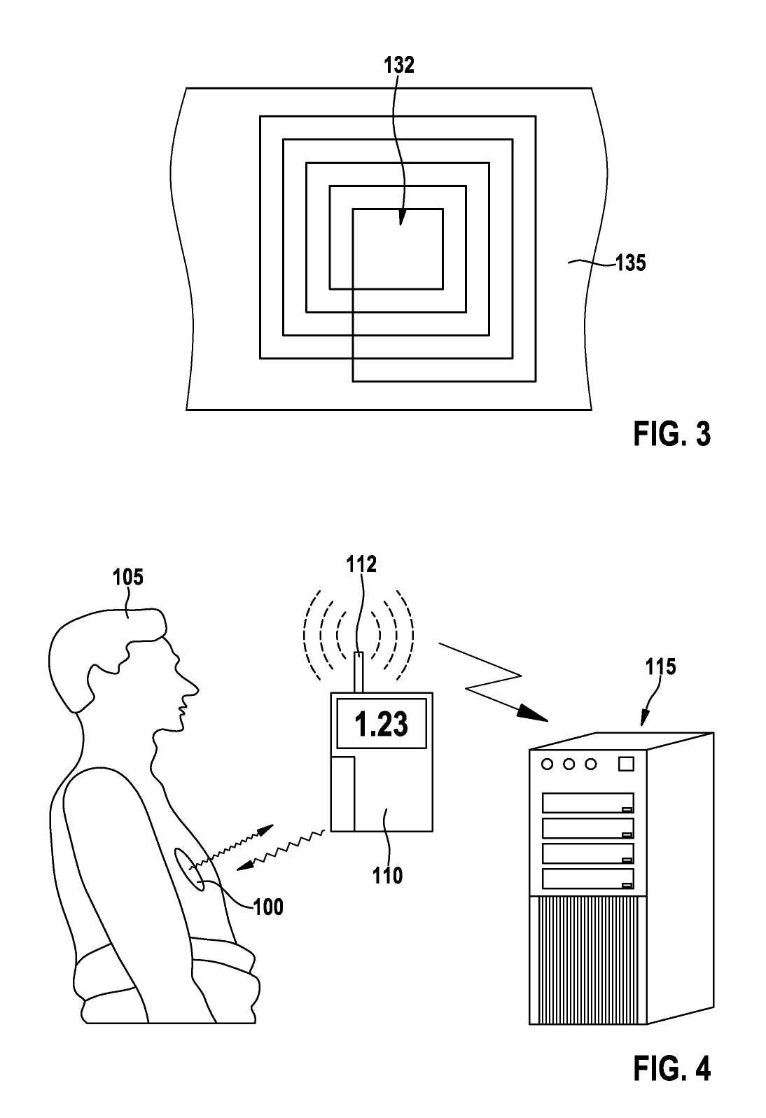

Instead of the coil 122 comprising strip electrodes 123 shown in FIG. 2, the planar coil 132 shown in FIG. 3 can also be used in the electric resonator 121. The planar coil 132 is disposed on a film 135.

The ends of the planar coil 132 shown in FIG. 3 are connected to each other. In this case, the oscillating circuit is obtained from the inductance of the planar coil 132 and the stray capacitances between the conductor tracks of the planar coil 132. These respective stray capacitances are altered, as described above, by endothelial cells attaching to the surface of the implant that is provided with the planar coil 132, whereby the resonant frequency of the array is also altered.

The current duration of the anticoagulation therapy is selected such that endothelialization of the implant is ensured in all patients to as great an extent as possible. For safety reasons, this therapy is given over 6 to 12 months, and likely longer than necessary, which incurs unnecessary costs for the health care system.

If endothelialization can be continuously measured by means of an implant according to the invention, the anticoagulation therapy can be tailored better and therefore shortened, resulting in increased safety, by extending this therapy for individual problem patients, and in increased subjective safety of the patients.

The solution according to the invention comprises only a passive sensor element and no battery and no electronics. It is simple, has a long service life, and does not change the dimensions of the implant. It enables wireless scanning without intervention. The implant according to the invention is only insignificantly more expensive as compared to the prior art because the microsensor can be produced in a cost-effective manner. It allows the progression of endothelialization to be detected during the healing process, without the involvement of the patent and physician, and it allows the data that is obtained to be evaluated mechanically and the persons involved to be automatically notified, if needed.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE NUMERALS

| | |
|---|---|
| 100 | Stent |
| 101 | Base body of the implant 100 |
| 103 | SAW element |
| 104 | Antenna |
| 105 | Human body |
| 110 | Scanning unit |
| 112 | Antenna |
| 115 | Processor |
| 121 | Electric resonator |
| 122 | Coil |
| 123 | Electrode |
| 125 | Film |
| 126 | Coating |
| 132 | Planar coil |
| 135 | Film |
| a | Thickness of film 125 |
| b | Thickness of electrode 123 |
| c | Thickness of coating 126 above electrode 123 |
| d | Width of electrode 123 |
| e | Width of space between electrodes 123 |

What is claimed is:

1. An implant having a base body that is optionally hollow and cylindrical, the implant comprising a device capable of measuring a degree of endothelialization and disposed on and/or in the base body, wherein the device comprises an acoustic resonator and/or an electric resonator.

2. The implant according to claim 1, characterized in that the acoustic resonator and/or the electric resonator are disposed such that a change in mass and/or surface area of coverage of a surface of the device by endothelial cells affects a corresponding change in an electric output signal of the device.

3. An implant according to claim 1, characterized in that the acoustic resonator comprises at least one surface acoustic wave (SAW) element.

4. An implant according to claim 1, characterized in that the electric resonator comprises at least one capacitor and at least one coil.

5. An implant according to claim 1, characterized in that the surface of the device comprises at least one coating.

6. An implant according to claim 1, characterized in that the device comprises an antenna.

7. An implant according to claim 1, characterized in that the implant comprises a pharmaceutically active substance on at least a portion of a surface of the base body thereof.

8. A system, comprising a catheter having a balloon and an implant according to claim 1, characterized in that the implant is disposed on the balloon of the catheter.
